(19) United States
(12) Patent Application Publication
(10) Pub. No.: US 2020/0281242 A1
(43) Pub. Date: Sep. 10, 2020

(54) COMPOSITION OF DIETARY SUPPLEMENTS AND METHODS OF MAKING THEREOF

(71) Applicants: Nantawanchaya Pajitpapan, Bang Kapi (TH); Sangchai Haelerttrukul, Mueang (TH)

(72) Inventors: Nantawanchaya Pajitpapan, Bang Kapi (TH); Sangchai Haelerttrukul, Mueang (TH)

(21) Appl. No.: 16/294,808

(22) Filed: Mar. 6, 2019

(51) Int. Cl. A23L 33/105; A23L 33/145; A61K 36/07; A61K 36/062; A61K 36/899; A61K 36/90; A61K 36/752; A61K 36/424; A61K 31/716; A61K 35/02

(52) U.S. Cl. CPC A23L 33/105; A61K 9/4833; A61K 36/07; A61K 36/062; A61K 36/899; A61K 36/90; A61K 36/752; A61K 36/424; A61K 31/716; A61K 35/02; A23V 2002/00; A23V 2200/324; A23V 2250/208; A23V 2250/218; A23V 2250/21162; A23V 2250/5034; A23L 33/145

(57) ABSTRACT

Disclosed are dietary supplement compositions and methods of making the dietary supplements in capsule form. For example, dietary supplement composition having Oryza sativa Linn, Cordyceps militaris, Ardisia polycephala Wall, Smilax corbularia Kunth, Smilax glabra Rhizome, cereal enzymes, Fomes rimosus (Berk.) Cooke, and edible bird's nest are provided.

### BACKGROUND OF THE INVENTION

### 1. Field of the Invention

The exemplary embodiment(s) of the present invention generally relates to the immune system and restoring health. More specifically, the exemplary embodiment(s) of the present invention relates to compositions of dietary supplements and the methods of making thereof.

### 2. Background

The immune system is important as it protects the human body from foreign viruses and bacteria. Without a strong immune system, a person is constantly ill and making it difficult to live a life filled with good physical health combined with a strong mind. There is a search of dietary supplements that have positive pharmacological affects in health promotion, balancing the immune system, and nourishing the human body.

Additionally, many dietary supplements are not certified for GMP quality assurance systems, which are guidelines for the product of dietary supplements. Further, many dietary supplements lack a hazard analysis and critical control points (HACCP), which can cause the final dietary supplement to be unsafe and increase safety risks due to harmful microorganisms, chemicals and various foreign matter.

Accordingly, there is a need to create a dietary supplement in capsule form that comprises photochemical contents that promote health, balance the immune system, nourish the human body, and that is certified for quality and safety.

### SUMMARY

According to an embodiment of the present invention, there is a dietary supplement comprising at least eight ingredients, including Oryza sativa linn, at least two fungi, Ardisia polycephala wall, Smilax corbularia kunth, Smilax glabra rhizome, cereal enzymes, and edible bird's nest, according to an embodiment of the present invention.

According to an embodiment of the present invention, there is a dietary supplement comprising at least eleven ingredients, including yeast beta-glucan, citrus bioflavonoid powder, rice extract, reishi extract, Cordyceps sinensis extract, at least two mushrooms, at least two fruits, gynostemma extract, and emblica extract, according to an embodiment of the present invention.

In further embodiments of the present invention, there is a method of making a dietary supplement comprising at least eight ingredients, according to an embodiment of the present invention.

According to another embodiment of the present invention, there is a method of making a dietary supplement comprising at least eight ingredients, according to an embodiment of the present invention.

According to another embodiment of the present invention, there is a method of making a dietary supplement comprising at least eleven ingredients, according to an embodiment of the present invention.

These features, advantages and other embodiments of the present invention are further made apparent, in the remainder of the present document, to those of ordinary skill in the art.

### BRIEF DESCRIPTION OF THE DRAWINGS

In order to more fully describe embodiments of the present invention, reference is made to the accompanying drawings. These drawings are not to be considered limitations in the scope of the invention, but are merely illustrative.

FIG. 1 illustrates a method of making dietary supplements in capsule form, according to an embodiment of the present invention.

### DETAILED DESCRIPTION OF SPECIFIC EMBODIMENTS

The description above and below and the drawings of the present document focus on one or more currently preferred embodiments of the present invention and also describe some exemplary optional features and/or alternative embodiments of the present invention. The description and drawings are for the purpose of illustration and not limitation. Those of ordinary skill in the art would recognize variations, modifications, and alternatives. Such variations, modifications, and alternatives are also within the scope of the present invention. Section titles are terse and are for convenience only.

Various embodiments of the present invention illustrated in the drawings may not be drawn to scale. Rather, the dimensions of the various features may be expanded or reduced for clarity. In addition, some of the drawings may be simplified for clarity. Thus, the drawings may not depict all of the components of a given apparatus (e.g., device) or method. Unless the meaning is clearly to the contrary, all ranges set forth herein are deemed to be inclusive of the endpoints.

This description provides dietary supplements useful for health promotion, balancing the immune system and nourishing the human body. The dietary supplement compositions provided herein can be in capsule or tablet form. The production of the dietary supplement, production equipment and dietary supplement storage are in accordance with regulation and good standard of production under general hygiene and the Ministry of Public Health. The dietary supplement is of good quality in respect to the notification of the Ministry of Public Health in regard to dietary supplements. The user of food additives is in accordance with notification of the Ministry of Public Health in regard to additive materials. The dietary supplement is acceptable by regulatory bodies, such as the Federal Drug Administration (FDA).

Oryza sativa Linn, polish rice bran used in the dietary supplement has been tested to have less than the maximum residue limit listed in the USP General Method for Pesticide Residues Analysis. Cordyceps militaris used in the dietary supplement has been tested to have less than the maximum residue limit listed in the USP General Method for Pesticide Residues Analysis. Fomes rimosus used in the dietary supplement has been tested to have less than the maximum residue limit listed in the USP General Method for Pesticide Residues Analysis. Smilax glabra Rhizome used in the dietary supplement has been tested to have less than the maximum residue limit listed in the USP General Method for Pesticide Residues Analysis.

In some cases, a dietary supplement composition can comprise rice, fungi, flowering plants, traditional medicine plant, herb, cereal enzymes, and edible bird's nest. In some cases, a dietary supplement composition can comprise eight ingredients such as, Oryza sativa Linn, Cordyceps militaris, Ardisia polycephala Wall, Smilax corbularia Kunth, Smilax glabra Rhizome, cereal enzymes, Fomes rimosus (Berk.) Cooke, and Swallow edible bird's nest.

An example of rice that can be included within a dietary supplement composition provided herein can be Oryza sativa Linn. Oryza sativa Linn is polish rice bran. Oryza sativa Linn can be purchased or isolated from a natural source. For example, at least 80 percent of a dietary supplement composition provided herein can be Oryza sativa Linn. In some cases, a dietary supplement composition provided herein can comprise about 400 mg. In some cases, a dietary supplement composition provided herein can be formulated to comprise an amount of Oryza sativa Linn such that a daily dose of about 400 mg to about 800 mg of Oryza sativa Linn can be conveniently consumed.

Examples of fungi that can be included within a dietary supplement composition provided herein include, without limitation, Fomes rimosus (Berk.) Cooke and Cordyceps militaris. In some cases, a dietary supplement composition can comprise one or more than one fugus. An example of fungus that can be included within a dietary supplement composition provided herein can be Fomes rimosus (Berk.) Cooke. Fomes rimosus (Berk.) Cooke is a mushroom. Fomes rimosus (Berk.) Cooke can be purchased or isolated from a natural source. For example, less than 1 percent of a dietary supplement composition provided herein can be Fomes rimosus (Berk.) Cooke. More specifically, about 0.4% of a dietary supplement composition provided herein can be Fomes rimosus (Berk.) Cooke. In some cases, a dietary supplement composition provided herein can comprise about 2 mg. In some cases, a dietary supplement composition provided herein can be formulated to comprise an amount of Fomes rimosus (Berk.) Cooke, such that a daily dose of about 2 mg to about 4 mg of Fomes rimosus (Berk.) Cooke can be conveniently consumed.

Another example of a fungus that can be included within a dietary supplement composition provided herein can be Cordyceps militaris. It can also be called Cordyceps militaris (Linn.), Link Mushroom for Rent Gold. Cordyceps militaris can be purchased or isolated from a natural source. For example, at least 4 percent of a dietary supplement composition provided herein can be Cordyceps militaris. In some cases, a dietary supplement composition provided herein can comprise about 20 mg. In some cases, a dietary supplement composition provided herein can be formulated to comprise an amount of Cordyceps militaris such that a daily dose of about 20 mg to about 40 mg of Cordyceps militaris can be conveniently consumed.

An example of a genus of flowering plants that can be included within a dietary supplement composition provided herein can be Ardisia polycephala Wall. Ardisia polycephala Wall is a flowering plant. Ardisia polycephala Wall can be purchased or isolated from a natural source. For example, at least 4 percent of a dietary supplement composition provided herein can be Ardisia polycephala Wall. In some cases, a dietary supplement composition provided herein can comprise about 20 mg. In some cases, a dietary supplement composition provided herein can be formulated to comprise an amount of Ardisia polycephala Wall such that a daily dose of about 20 mg to about 40 mg of Ardisia polycephala Wall can be conveniently consumed.

An example of a traditional medicine plant that can be included within a dietary supplement composition provided herein can be Smilax corbularia Kunth. Smilax corbularia Kunth is a traditional medicine plant. Smilax corbularia Kunth can be obtained by isolating the rhizomes of the plant or it can be purchased. For example, at least 4 percent of a dietary supplement composition provided herein can be Smilax corbularia Kunth. In some cases, a dietary supplement composition provided herein can comprise about 20 mg. In some cases, a dietary supplement composition provided herein can be formulated to comprise an amount of Smilax corbularia Kunth such that a daily dose of about 20 mg to about 40 mg of Smilax corbularia Kunth can be conveniently consumed.

An example of an herb that can be included within a dietary supplement composition provided herein can be Smilax glabra Rhizome. Smilax glabra Rhizome is also known as Sarsaparilla and it is a traditional medicine plant. Smilax glabra Rhizome can be obtained by isolating the rhizomes of the plant or it can be purchased. For example, at least 4 percent of a dietary supplement composition provided herein can be Smilax glabra Rhizome. In some cases, a dietary supplement composition provided herein can comprise about 20 mg. In some cases, a dietary supplement composition provided herein can be formulated to comprise an amount of Smilax glabra Rhizome such that a daily dose of about 20 mg to about 40 mg of Smilax glabra Rhizome can be conveniently consumed.

Cereal enzymes are used to facilitate processing and to achieve improved and uniform product quality. Cereal enzymes can be purchased or isolated from a natural source. For example, at least 3 percent of a dietary supplement composition provided herein can be cereal enzymes. In some cases, a dietary supplement composition provided herein can comprise about 16 mg of cereal enzymes. In some cases, a dietary supplement composition provided herein can be formulated to comprise an amount of cereal enzymes such that a daily dose of about 16 mg to about 32 mg of cereal enzymes can be conveniently consumed.

An example of an edible bird's nest that can be included within a dietary supplement composition provided herein can be from the Swallow. The Swallow edible bird's nest is a nest made by the bird's saliva. The Swallow edible bird's nest can be purchased or isolated from a natural source. For example, less than 1 percent of a dietary supplement composition provided herein can be Swallow edible bird's nest. More specifically, about 0.4% of a dietary supplement composition provided herein can be Swallow edible bird's nest. In some cases, a dietary supplement composition provided herein can comprise about 2 mg. In some cases, a dietary supplement composition provided herein can be formulated to comprise an amount of Swallow edible bird's nest such that a daily dose of about 2 mg to about 4 mg of the Swallow edible bird's nest can be conveniently consumed.

In other cases, a dietary supplement composition can comprise beta-glucan, flavonoids, rice extract, mushroom extract, mushroom powder, fruit powder, herb extract, and natural plant extract.

In yet other cases, a dietary supplement composition can comprise eleven active ingredients, such as, yeast beta-glucan, citrus bioflavonoid powder, rice extract, reishi extract, Cordyceps sinensis extract, maitake mushroom powder, shiitake mushroom powder, pineapple powder, papaya powder, gynostemma extract, and emblica extract. In addition, there can be two inactive ingredients such as titanium dioxide and gelatin 428. This embodiment of the dietary supplement composition can be in the form of a capsule with a net weight for one capsule at about 500-600 milligrams. For example, the net weight for one capsule can be 500 milligrams with a preferred dosage of two capsules per day. In another example, the net weight for one capsule can be 600 milligrams with a preferred dosage of two capsules per day.

An example of beta-glucan that can be included within a dietary supplement composition provided herein can be yeast beta-glucan. Yeast beta-glucan can be purchased or isolated from a natural source. For example, a range of about 16 to 20 percent of a dietary supplement composition provided herein can be yeast beta-glucan. In some cases, a dietary supplement composition provided herein can comprise about 100 mg. In some cases, a dietary supplement composition provided herein can be formulated to comprise an amount of yeast beta-glucan such that a daily dose of about 100 mg to about 200 mg of yeast beta-glucan can be conveniently consumed.

An example of flavonoids that can be included within a dietary supplement composition provided herein can be citrus bioflavonoid. Citrus bioflavonoid can be in powder form. Citrus bioflavonoid powder can be purchased or isolated from a natural source. For example, a range of about 8 to 10 percent of a dietary supplement composition provided herein can be Citrus bioflavonoid powder. In some cases, a dietary supplement composition provided herein can comprise about 50 mg. In some cases, a dietary supplement composition provided herein can be formulated to comprise an amount of Citrus bioflavonoid powder such that a daily dose of about 50 mg to about 100 mg of Citrus bioflavonoid powder can be conveniently consumed.

Rice extract can be purchased or isolated from a natural source. For example, a range of about 8 to 10 percent of a dietary supplement composition provided herein can be rice extract. In some cases, a dietary supplement composition provided herein can comprise about 50 mg. In some cases, a dietary supplement composition provided herein can be formulated to comprise an amount of rice extract such that a daily dose of about 50 mg to about 100 mg of rice extract can be conveniently consumed.

Examples of mushroom extract that can be included within a dietary supplement composition provided herein include, without limitation, reishi extract and Cordyceps sinensis extract. In some cases, a dietary supplement composition can comprise one or more than one mushroom extract. An example of mushroom extract that can be included within a dietary supplement composition provided herein can be reishi extract. Reishi extract can be purchased or isolated from a natural source. For example, a range of about 8 to 10 percent of a dietary supplement composition provided herein can be reishi extract. In some cases, a dietary supplement composition provided herein can comprise about 50 mg. In some cases, a dietary supplement composition provided herein can be formulated to comprise an amount of reishi extract such that a daily dose of about 50 mg to about 100 mg of reishi extract can be conveniently consumed.

In some cases, the mushroom extract of a dietary supplement composition provided herein can be Cordyceps sinensis extract. Cordyceps sinensis extract can be purchased or isolated from a natural source. For example, a range of about 8 to 10 percent of a dietary supplement composition provided herein can be Cordyceps sinensis extract. In some cases, a dietary supplement composition provided herein can comprise about 50 mg. In some cases, a dietary supplement composition provided herein can be formulated to comprise an amount of Cordyceps sinensis extract such that a daily dose of about 50 mg to about 100 mg of Cordyceps sinensis extract can be conveniently consumed.

Examples of mushroom that can be included within a dietary supplement composition provided herein include, without limitation, maitake mushroom and shiitake mushroom. In some cases, a dietary supplement composition can comprise one or more than one mushroom.

An example of a mushroom that can be included within a dietary supplement composition provided herein can be maitake mushroom. Maitake mushroom can be in powder form. Maitake mushroom powder can be purchased or isolated from a natural source. For example, a range of about 6 to 8 percent of a dietary supplement composition provided herein can be maitake mushroom powder. In some cases, a dietary supplement composition provided herein can comprise about 40 mg. In some cases, a dietary supplement composition provided herein can be formulated to comprise an amount of maitake mushroom powder such that a daily dose of about 40 mg to about 80 mg of maitake mushroom powder can be conveniently consumed.

Another example of a mushroom that can be included within a dietary supplement composition provided herein can be shiitake mushrooms. Shiitake mushroom can be in powder form. Shiitake mushroom powder can be purchased or isolated from a natural source. For example, a range of about 6 to 8 percent of a dietary supplement composition provided herein can be shiitake mushroom powder. In some cases, a dietary supplement composition provided herein can comprise about 40 mg. In some cases, a dietary supplement composition provided herein can be formulated to comprise an amount of shiitake mushroom powder such that a daily dose of about 40 mg to about 80 mg of shiitake mushroom powder can be conveniently consumed.

Examples of fruit powder that can be included within a dietary supplement composition provided herein include, without limitation, pineapple powder and papaya powder. In some cases, a dietary supplement composition can comprise one or more than one fruit powder.

An example of fruit powder that can be included within a dietary supplement composition provided herein can be pineapple powder. Pineapple powder can be purchased or isolated from a natural source. For example, a range of about 6 to 8 percent of a dietary supplement composition provided herein can be pineapple powder. In some cases, a dietary supplement composition provided herein can comprise about 40 mg. In some cases, a dietary supplement composition provided herein can be formulated to comprise an amount of pineapple powder such that a daily dose of about 40 mg to about 80 mg of pineapple powder can be conveniently consumed.

Another example of a fruit powder that can be included within a dietary supplement composition provided herein can be papaya powder. Papaya powder can be purchased or isolated from a natural source. For example, a range of about 5 to 7 percent of a dietary supplement composition provided herein can be papaya powder. In some cases, a dietary supplement composition provided herein can comprise about 35 mg. In some cases, a dietary supplement composition provided herein can be formulated to comprise an amount of papaya powder such that a daily dose of about 35 mg to about 70 mg of papaya powder can be conveniently consumed.

An example of an herb extract that can be included within a dietary supplement composition provided herein can be gynostemma extract. Gynostemma extract can be purchased or isolated from a natural source. For example, a range of about 4 to 5 percent of a dietary supplement composition provided herein can be gynostemma extract. In some cases, a dietary supplement composition provided herein can comprise about 25 mg. In some cases, a dietary supplement composition provided herein can be formulated to comprise an amount of gynostemma extract such that a daily dose of about 25 mg to about 50 mg of gynostemma extract can be conveniently consumed.

An example of a natural plant extract that can be included within a dietary supplement composition provided herein can be emblica extract. Emblica extract can be purchased or isolated from a natural source. For example, a range of about 3 to 4 percent of a dietary supplement composition provided herein can be emblica extract. In some cases, a dietary supplement composition provided herein can comprise about 20 mg. In some cases, a dietary supplement composition provided herein can be formulated to comprise an amount of emblica extract such that a daily dose of about 20 mg to about 40 mg of emblica extract can be conveniently consumed.

Dietary supplement compositions provided herein can be formulated for oral administration and can include suitable excipients, flavorings, colorants, and other ingredients. For oral administration, capsules can be prepared with pharmaceutically acceptable excipients such as binding agents, fillers, lubricants, disintegrants, or wetting agents. Preparations for oral administration can be suitably formulated to give controlled release of one or more compounds.

In some cases, dietary supplement compositions provided herein can contain a pharmaceutically acceptable carrier for administration to a human, including, without limitation, sterile aqueous, or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Pharmaceutically acceptable carriers also can include physiologically acceptable aqueous vehicles (e.g., physiological saline) or other carriers appropriate for oral administration.

In some cases, the dietary supplement compositions provided herein can be in the form of a capsule, by way of an example only, configured to have a unit dosage equal to the daily desired dosage. For example, if a human desires 500 mg of a particular agent, each capsule can include about 500 mg in weight of that agent. The dosages of a particular dietary supplement compositions provided herein will depend on many factors including the general health of the human. For instance, in some cases, an exemplary dietary supplement composition can be provided in two separate capsules.

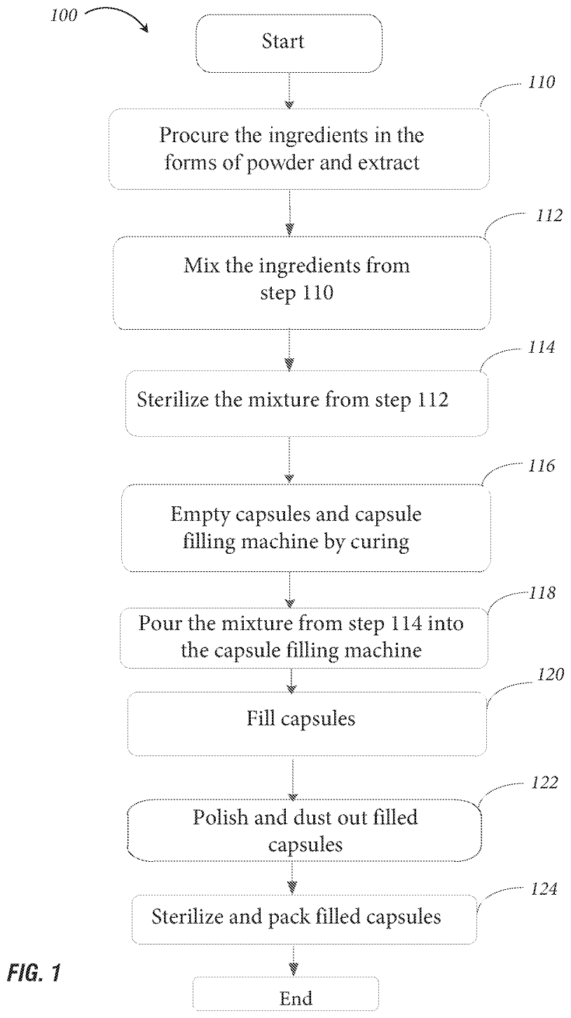

Referring now to FIG. 1, which illustrates a method of making dietary supplements in capsule form 100, according to an embodiment of the present invention. In step 110, when making the eight ingredients dietary supplement capsule, the eight ingredients in the forms of powder and extract are procured as mentioned above. In step 112, the eight ingredients from step 110 are mixed. In step 114, the mixture from step 112 is sterilized. In step 116, the capsules and the capsule filling machine are emptied by curing. In step 118, the mixture from step 114 is poured into the capsule filling machine. In step 120, the capsules are filled. In step 122, the filled capsules are polished and dusted. In step 124, the filled capsules from step 122 are sterilized and packed.

The method of making the eleven ingredient dietary supplement capsule is similar to a method of making dietary supplements in capsule form 100; however, for step 110, the ingredients for the eleven ingredient capsule as mentioned above are used instead.

The invention will be further described in the following examples, which do not limit the scope of the invention described in the claims.

### EXAMPLES

### Example 1

An Exemplary Dietary Supplement Composition

A dietary supplement was prepared with the composition shown in Table 1 below. The active ingredients in 1 capsule (500 mg).

TABLE 1
Dietary Supplement composition

| | | |
|---|---|---|
| Oryza sativa Linn | 400 | mg |
| Cordyceps militaris | 20 | mg |
| Ardisia polycephala Wall | 20 | mg |
| Smilax corbularia Kunth | 20 | mg |
| Smilax Glabra Rhizome | 20 | mg |
| cereal enzymes | 16 | mg |
| Fomes rimosus (Berk.) Cooke | 2 | mg |
| edible bird's nest | 2 | mg |

### Example 2

An Exemplary Dietary Supplement Composition

A dietary supplement can be prepared with the composition shown in Table 2 below. The active ingredients in 1 capsule (500 mg)

TABLE 2
Dietary Supplement composition

| | | |
|---|---|---|
| yeast beta-glucan | 100 | mg |
| citrus bioflavonoid powder | 50 | mg |
| rice extract | 50 | mg |
| reishi extract | 50 | mg |
| cordyceps sinensis extract | 50 | mg |
| maitake mushroom powder | 40 | mg |
| shiitake mushroom powder | 40 | mg |
| pineapple powder | 40 | mg |
| papaya powder | 35 | mg |
| gynostemma extract | 25 | mg |
| emblica extract | 20 | mg |

### Example 3

An Exemplary Dietary Supplement Composition

A dietary supplement can be prepared with the composition shown in Table 3 below. The active ingredients in 1 capsule (600 mg)

TABLE 3
Dietary Supplement composition

| | | |
|---|---|---|
| yeast beta-glucan | 100 | mg |
| citrus bioflavonoid powder | 50 | mg |
| rice extract | 50 | mg |
| reishi extract | 50 | mg |
| cordyceps sinensis extract | 50 | mg |
| maitake mushroom powder | 40 | mg |
| shiitake mushroom powder | 40 | mg |
| pineapple powder | 40 | mg |
| papaya powder | 35 | mg |
| gynostemma extract | 25 | mg |
| emblica extract | 20 | mg |

While particular embodiments of the present invention have been shown and described, it will be obvious to those of skills in the art that based upon the teachings herein, changes and modifications may be made without departing from this exemplary embodiment(s) of the present invention and its broader aspects. Therefore, the appended claims are intended to encompass within their scope all such changes and modifications as are within the true spirit and scope of this exemplary embodiment(s) of the present invention.

Throughout the description and drawings, example embodiments of the present invention are given with reference to specific configurations. It will be appreciated by those of ordinary skill in the art that the present invention can be embodied in other specific forms. Those of ordinary skill in the art would be able to practice such other embodiments of the present invention without undue experimentation. The scope of the present invention, for the purpose of the present patent document, is not limited merely to the specific example embodiments of the present invention or alternatives of the foregoing description.

What is claimed is:

1. A composition useful for health promotion comprising:
(a) about 100 mg of yeast beta-glucan,
(b) about 50 mg of citrus bioflavonoid powder,
(c) about 50 mg of rice extract,
(d) about 50 mg of reishi extract,
(e) about 50 mg of Cordyceps sinensis extract,
(f) at least two mushrooms,
(g) at least two fruits,
(h) about 25 mg of gynostemma extract, and
(i) about 20 mg of emblica extract.

2. The composition of claim 1, wherein the at least two mushrooms comprise a maitake mushroom powder.

3. The composition of claim 1, wherein the at least two mushrooms comprise a shiitake mushroom powder.

4. The composition of claim 2, wherein the maitake mushroom powder is about 40 mg.

5. The composition of claim 3, wherein the shiitake mushroom powder is about 40 mg.

6. The composition of claim 1, wherein the at least two fruits comprise a pineapple powder.

7. The composition of claim 1, wherein the at least two fruits comprise a papaya powder.

8. The composition of claim 6, wherein the pineapple power is about 40 mg.

9. The composition of claim 7, wherein the papaya powder is about 35 mg.

10. The composition of claim 1, wherein the composition is in a form of a capsule.

11. A composition useful for health promotion comprising:
(a) about 400 mg of Oryza sativa linn,
(b) at least two fungi,
(c) about 20 mg of Ardisia polycephala wall,
(d) about 20 mg of Smilax corbularia kunth,
(e) about 20 mg of Smilax glabra rhizome,
(f) about 16 mg of cereal enzymes, and
(g) about 2 mg of edible bird's nest.

12. The composition of claim 11, wherein the at least two fungi comprise a Fomes rimosus cooke.

13. The composition of claim 12, wherein the Fomes rimosus cooke is about 2 mg.

14. The composition of claim 11, wherein the at least two fungi comprise a Cordyceps militaris.

15. The composition of claim 14, wherein the Cordyceps militaris is about 20 mg.

16. The composition of claim 11, wherein the edible bird's nest is from a swallow.

17. The composition of claim 11, wherein the composition is in a form of a capsule.

18. A method of manufacturing a composition useful for health promotion, comprising the steps of:
(a) mixing active ingredients,
(b) sterilizing the mixture from step (a),
(c) pouring the mixture from step (b) into a capsule filling machine,
(d) filling capsules, and
(e) sterilizing the filled capsules from step (d).

19. The method of manufacturing a composition of claim 18, wherein the active ingredients are Oryza sativa linn, Fomes rimosus cooke, Cordyceps militaris, Ardisia polycephala wall, Smilax corbularia kunth, Smilax glabra rhizome, cereal enzymes, and edible bird's nest.

20. The method of manufacturing a composition of claim 18, wherein the active ingredients are yeast beta-glucan, citrus bioflavonoid powder, rice extract, reishi extract, Cordyceps sinensis extract, maitake mushroom powder, shiitake mushroom powder, pineapple powder, papaya powder, gynostemma extract, and emblica extract.
